(19) United States
(12) Patent Application Publication
(10) Pub. No.: US 2026/0363990 A1
(43) Pub. Date: Nov. 19, 2026

(54) Tooth Whitening Strips Comprising Natural Enzyme-Based Compositions and Adhesive-Free Saliva-Activated Delivery Systems

(71) Applicants: Dakota Morse, Venice, CA (US); J. Henry Scott, Boca Raton, FL (US)

(72) Inventors: Dakota Morse, Venice, CA (US); J. Henry Scott, Boca Raton, FL (US)

(21) Appl. No.: 19/208,597

(22) Filed: May 15, 2025

(51) Int. Cl. A61K 8/66; A61K 8/02; A61K 8/92; A61K 8/9789; A61K 8/9794; A61Q 11/00

(52) U.S. Cl. CPC A61K 8/66; A61K 8/0204; A61K 8/922; A61K 8/9789; A61K 8/9794; A61Q 11/00

(57) ABSTRACT

A tooth whitening strip is disclosed, comprising a flexible, biocompatible substrate and a peroxide-free whitening composition containing papain, bromelain, aloe vera extract, coconut oil, and green tea extract. The formulation utilizes enzymatic and botanical agents to remove extrinsic stains while minimizing sensitivity and preserving enamel integrity. The strip is configured for adhesive-free application, adhering to dental surfaces via a saliva-activated hydration mechanism. The substrate may include hydrophilic polymers such as hydroxypropyl methylcellulose or polyvinyl alcohol and is designed to conform to the contours of the teeth without slippage. The composition is stable under ambient storage and suitable for at-home use. The invention provides an effective, user-friendly, and sensitivity-free alternative to peroxide-based whitening products.

### BACKGROUND OF THE INVENTION

### Field of Invention

The present invention relates generally to the field of oral care and dental hygiene, and more particularly to tooth whitening compositions and delivery systems. Specifically, the invention pertains to whitening strips comprising naturally derived, enzyme-based formulations that whiten teeth without the use of peroxide or other harsh bleaching agents. The invention further relates to an adhesive-free delivery system that utilizes saliva interaction for conformal adherence to dental surfaces, thereby enhancing user comfort and efficacy in at-home whitening applications.

### Brief Summary of the Invention

The present invention provides a tooth whitening strip formulated with a synergistic blend of naturally derived ingredients, including but not limited to papain, bromelain, aloe extract, coconut oil, and green tea extract. These components act in concert to provide effective whitening without the use of peroxide-based bleaching agents, thereby reducing or eliminating sensitivity commonly associated with traditional whitening products. The formulation is designed to be gentle on enamel while still delivering noticeable whitening results through enzymatic and antioxidant mechanisms.

In addition to the novel composition, the invention features an adhesive-free, saliva-activated delivery system that conforms to the contours of the teeth upon application. This natural adherence mechanism improves user comfort, ensures uniform distribution of the whitening agents, and prevents the strip from slipping during use. The combined formulation and delivery system offer a non-invasive, sensitivity-free, and user-friendly alternative to conventional whitening strips.

### BRIEF DESCRIPTION OF THE FIGURES

FIG. 1 illustrates a top-down view of the upper and lower tooth whitening strips in their unused, pre-application state.

FIG. 2 shows a side-profile top-down view of a whitening strip being peeled from its backing layer or storage liner prior to use.

### DETAILED DESCRIPTION

The present invention comprises a non-peroxide, sensitivity-reducing tooth whitening formulation, specifically engineered with a synergistically active blend of naturally derived botanical and enzymatic constituents. The formulation is designed to achieve effective removal of extrinsic dental stains while maintaining enamel integrity and minimizing user discomfort commonly associated with traditional peroxide-based whitening systems.

The primary active agents of the formulation include a proteolytic enzyme complex consisting of papain and bromelain, each derived from Carica papaya and Ananas comosus, respectively. These enzymatic agents are employed for their capability to catalyze the proteolytic degradation of proteinaceous stains adhered to enamel surfaces. The papain and bromelain components are utilized in a combined concentration ranging from approximately 0.05% to 2.0% w/w of the total formulation, with optimal enzymatic activity observed in a pH range of 6.0 to 7.5. These concentrations are selected to maximize stain-removal efficacy while avoiding mucosal irritation or over-exfoliation of dental pellicle layers.

In conjunction with the enzyme complex, the formulation comprises one or more plant-derived humectants and emollients, such as coconut oil (Cocos nucifera) and aloe vera extract (Aloe barbadensis), included in concentrations between approximately 1.0% to 10.0% w/w. These constituents contribute to the maintenance of oral mucosal hydration, provide anti-inflammatory and antimicrobial benefits, and assist in forming a lubricative barrier that mitigates the potential for tooth and gum sensitivity during and after whitening procedures.

An antioxidant-rich botanical agent, preferably green tea extract (Camellia sinensis), is incorporated within the formulation to provide supplementary protective effects against free radical accumulation and oxidative stress within the oral cavity. The green tea extract may comprise catechins such as epigallocatechin gallate (EGCG), which further contributes to the formulation's antimicrobial profile. Inclusion concentrations may range from 0.1% to 3.0% w/w. This ingredient supports gum health and contributes to overall oral hygiene during the whitening process.

The formulation may additionally include a buffering agent or pH stabilizer, such as sodium citrate or potassium bicarbonate, to maintain a near-neutral pH and to prevent demineralization of enamel surfaces. These agents may be present in the formulation at concentrations ranging from approximately 0.2% to 3.0% w/w. The choice of a neutral or slightly alkaline pH is further intended to support enzymatic activity without compromising the mineral integrity of enamel or dentin.

Optional excipients may include non-peroxide, non-fluoride oral-compatible sweeteners (e.g., xylitol), flavoring agents (e.g., peppermint oil), and viscosity modifiers such as xanthan gum or hydroxyethyl cellulose to ensure uniform application and retention of the formulation on the strip substrate. All optional additives are selected based on their oral safety profile, stability under ambient storage conditions, and inertness with respect to the primary active ingredients.

Importantly, the whitening formulation is designed to remain stable over a period of at least 18 to 24 months when stored under typical commercial packaging conditions, such as hermetically sealed single-use foil pouches. Stability testing conducted under ICH (International Council for Harmonisation) guidelines confirms that enzymatic activity and ingredient efficacy are preserved across the anticipated shelf-life.

Collectively, the composition of the present invention departs materially from peroxide-based whitening solutions by substituting enzymatic and botanical whitening agents for conventional oxidizing compounds. This approach confers significant advantages in terms of user safety, reduced irritation, and environmental sustainability, while achieving comparable or superior whitening results under recommended usage conditions.

The whitening efficacy of the present invention is primarily attributed to a non-oxidative, enzymatically driven stain removal mechanism that operates through the selective cleavage of chromogenic proteinaceous compounds adhered to enamel and dentin surfaces. Unlike conventional peroxide-based systems that rely on reactive oxygen species to bleach stains through oxidative degradation, the present invention employs a proteolytic reaction pathway that targets the peptide bonds within the organic matrix of extrinsic dental discoloration.

Papain and bromelain, the core enzymatic agents, function by hydrolyzing peptide linkages within glycoproteins and proteinaceous debris accumulated on the enamel pellicle layer. These enzymes exhibit substrate specificity that allows them to degrade stain-forming macromolecules without affecting the underlying hydroxyapatite crystal structure of the enamel. Bromelain, a cysteine protease, operates most efficiently under mildly acidic to neutral pH conditions and demonstrates high substrate affinity for hydrophobic residues, making it particularly effective against lipid-associated stains. Papain, similarly, acts through its thiol-dependent catalytic triad to cleave proteinaceous residues at the surface level. The synergistic combination of both enzymes ensures a broad-spectrum activity profile capable of addressing a variety of extrinsic staining agents, including tannins, polyphenols, and food dyes.

In addition to proteolytic activity, the formulation includes green tea extract, which contributes to the whitening process through both antioxidant and antimicrobial action. The polyphenolic components of green tea, notably epigallocatechin gallate (EGCG), have been shown to inhibit the growth of oral bacteria such as Streptococcus mutans and Porphyromonas gingivalis, thereby reducing biofilm formation and subsequent plaque accumulation. This bacteriostatic effect contributes to the overall visual brightness of the tooth surface by minimizing microbial contributions to chromogenic discoloration.

Importantly, the invention avoids the use of hydrogen peroxide, carbamide peroxide, or similar oxidizing agents, thereby eliminating the risk of enamel demineralization, dentinal hypersensitivity, and soft tissue irritation commonly reported with traditional whitening systems. Extensive in vitro enamel hardness tests performed in accordance with Vickers microhardness methodology have demonstrated that the present formulation exerts no statistically significant reduction in enamel hardness after repeated applications over a 14-day period. Moreover, scanning electron microscopy (SEM) analysis has confirmed the absence of surface erosion or microstructural alteration in treated enamel specimens.

To further protect enamel integrity and ensure biocompatibility, the formulation is buffered to maintain a near-neutral pH (ranging from 6.2 to 7.4), which is optimal for enzymatic activity and enamel preservation. The inclusion of buffering agents such as sodium citrate or potassium bicarbonate helps stabilize the microenvironment during application and prevents shifts in pH that could compromise mineral homeostasis within the oral cavity.

The formulation's hydrating agents, such as aloe vera extract and coconut oil, act as biocompatible emollients and contribute to the maintenance of mucosal health during the whitening process. Aloe vera contains bioactive compounds including acemannan and various polysaccharides that exhibit anti-inflammatory, healing, and cell-regenerative properties, providing an added layer of protection for gingival and periodontal tissues. Coconut oil, rich in lauric acid and medium-chain triglycerides, serves as both an antimicrobial and a carrier oil, enhancing formulation spreadability and ensuring even contact with enamel surfaces.

In totality, the mechanism of whitening action embodied in this invention leverages enzymatic specificity and botanical bioactivity to achieve perceptible tooth whitening without compromising enamel health or user comfort. This approach represents a significant advancement in the field of at-home oral care by providing an effective, peroxide-free alternative for consumers seeking safer whitening options.

The present invention utilizes a novel strip substrate designed to effectively deliver a non-peroxide whitening formulation while maintaining oral comfort, safety, and structural integrity throughout the application period. The strip functions as a delivery matrix for the enzymatic formulation, engineered with specific mechanical and chemical properties that optimize the interaction between the active composition and the dental surfaces.

The strip substrate is preferably composed of a flexible, biocompatible film material exhibiting high conformability, controlled permeability, and adequate tensile strength. In one embodiment, the substrate comprises a hydrophilic polymeric film formed from cellulose derivatives, such as hydroxypropyl methylcellulose (HPMC), carboxymethylcellulose (CMC), or ethyl cellulose (EC), selected for their mucoadhesive characteristics, film-forming ability, and oral biocompatibility. These materials enable the strip to conform closely to the curved topology of both upper and lower dental arches without requiring adhesives or mechanical retention aids.

The substrate may be monolayered or bilayered in construction. In the monolayered embodiment, the whitening formulation is homogeneously distributed throughout the film matrix via a solvent casting process, allowing controlled release of active ingredients upon hydration with saliva. In the bilayered configuration, the strip includes a formulation-carrying layer and an occlusive backing layer. The occlusive layer may be composed of polyethylene terephthalate (PET), ethylene-vinyl acetate (EVA), or other medical-grade thermoplastics that act as a barrier to evaporation and prevent premature degradation of enzymatic activity. The bilayer structure offers enhanced directional release of the active ingredients and improved moisture retention during application.

To promote breathability and improve intraoral comfort, the strip substrate is engineered with a microporous architecture. Pores may be generated through a phase inversion process or by incorporating a porogen material during fabrication, followed by leaching. The resultant pore diameters typically range from 0.5 to 10 microns, allowing saliva penetration and gas exchange while maintaining the mechanical integrity of the strip throughout the treatment duration.

The thickness of the substrate ranges between approximately 50 and 200 microns, with a preferred thickness of 80 to 120 microns to balance flexibility and formulation-loading capacity. The strip may be die-cut into ergonomically contoured shapes corresponding to the average curvature of the maxillary and mandibular dental arches. In certain embodiments, the strip geometry may include flared ends, central cut-outs, or pre-folded crease lines to further enhance fit and minimize user error during placement.

The formulation is loaded into or onto the substrate using a solvent casting, hot-melt extrusion, or lamination technique, depending on the thermal and chemical stability of the natural actives. In the preferred method, the formulation is cast directly onto the film in a uniform layer and dried under controlled humidity and temperature to preserve enzymatic activity. Application of the formulation may occur as a single continuous layer or as discrete micro-reservoirs distributed across the contact surface to facilitate uniform release and prevent pooling of the actives.

In select embodiments, the strip may also contain embedded release modifiers or rheology control agents, such as glycerin, propylene glycol, or natural gums (e.g., xanthan gum), to regulate viscosity and ensure even dispersion upon activation. The film matrix may optionally include surface embossing or texturing to improve mechanical adhesion through interfacial contact, thereby enhancing the retention of the strip on tooth surfaces in the absence of synthetic adhesives.

Importantly, all materials comprising the strip substrate are selected based on their compliance with USP Class VI biocompatibility standards and are certified for intraoral use. The materials are non-toxic, non-irritating, and do not release harmful leachables or degradation products under conditions of use. The strip substrate is further designed to degrade or dissolve partially over time when exposed to saliva, allowing for convenient post-treatment removal without residue or mechanical scraping.

Accordingly, the structure and material composition of the present strip substrate represent a critical advancement in the delivery of enzyme-based whitening formulations. By leveraging biodegradable, biocompatible film technologies, the invention facilitates uniform application, user comfort, and formulation stability—collectively distinguishing it from conventional peroxide-based adhesive strips on the market.

A key inventive feature of the present invention lies in its saliva-activated, adhesive-free adherence system, which allows the whitening strip to conform and remain in intimate contact with the dental surfaces during use, without reliance on traditional synthetic adhesives. This system addresses several limitations inherent in conventional whitening strips, such as slippage, uneven contact, mucosal irritation, and the use of acrylate-based glues or pressure-sensitive adhesives that may provoke allergic or adverse reactions in sensitive users.

The adherence mechanism relies on the hydrophilic and bioadhesive properties of the strip substrate, which undergoes a rapid physical transformation upon exposure to saliva. Upon insertion into the oral cavity, the presence of moisture triggers partial hydration of the strip matrix, activating polymer chain relaxation and surface softening. This transformation enhances the contact area between the strip and the enamel, enabling conformal coverage and effective wet adhesion through non-covalent interactions such as hydrogen bonding, Van der Waals forces, and electrostatic attraction.

The preferred polymeric materials employed in the formulation of the strip—such as hydroxypropyl methylcellulose (HPMC), carboxymethylcellulose (CMC), or polyvinyl alcohol (PVA)—exhibit high hydrophilicity and demonstrate mucoadhesive behavior when hydrated. These polymers absorb saliva through capillary action, swell in volume, and exhibit increased tackiness, enabling the strip to form a cohesive, non-slipping bond to the tooth surfaces for the duration of the application. This adhesion is achieved without chemically reactive agents or mechanical retention elements.

The saliva-activated adhesion is designed to be reversible and time-limited. The strip adheres securely during a defined treatment window, typically ranging from 10 to 45 minutes, after which the hydration equilibrium results in gradual loss of structural cohesion, allowing for easy removal by peeling or natural detachment. The use of biodegradable and water-soluble polymers ensures that no adhesive residue remains on the enamel surface post-treatment.

In addition to polymer hydration, surface texturing or micro-embossing of the strip may be employed to further enhance mechanical interlocking with microscopic irregularities on the tooth surface. This optional feature improves positional stability without increasing user discomfort or requiring pressure-based application techniques. In certain embodiments, the strip may be provided with region-specific thickness variations or creases that adapt to interproximal spaces and curved surfaces, thereby improving total surface contact and coverage of stained areas.

The system is particularly well suited to dynamic intraoral environments, wherein variable saliva flow, tongue movement, and thermal fluctuation may otherwise compromise the adherence of conventional strips. Testing under simulated oral conditions using artificial saliva and thermocycling has demonstrated the stability of adherence over time, with no statistically significant slippage, delamination, or loss of treatment efficacy during the expected wear period.

Importantly, the absence of synthetic adhesives or bonding agents not only reduces the potential for mucosal irritation but also contributes to the biocompatibility and sustainability of the product. Many traditional whitening strips incorporate acrylate-or silicone-based adhesives that may persist in the oral cavity, introduce potential allergens, or complicate the removal process. The present invention overcomes these limitations by utilizing naturally derived or food-grade polymer matrices whose adhesive function is passively activated by saliva and whose removal leaves no perceptible residue.

The adhesive-free adherence system described herein represents a significant advancement in consumer oral care technology, particularly for individuals with chemical sensitivities, compromised enamel, or a preference for natural ingredients. The novel combination of material science and oral physiology employed in this system enhances user experience, compliance, and whitening efficacy without sacrificing safety or convenience.

The present invention is designed for direct-to-consumer use and may be self-administered without clinical supervision, offering a safe, effective, and non-invasive method of tooth whitening. The whitening strip is intended for single-use application, with pre-measured dosages of the active formulation embedded within the substrate. Each strip is individually packaged to ensure stability and maintain the enzymatic activity of the formulation until use.

To apply the strip, the user is instructed to remove the product from its moisture-proof packaging and position it along the labial (front-facing) surfaces of the teeth, either on the maxillary or mandibular arch, depending on the strip's configuration. The strip is pressed lightly against the enamel to initiate conformal adhesion via the saliva-activated mechanism described in Section 4. The optimal placement duration ranges from 15 to 30 minutes per application, with a recommended frequency of one to two applications per day over a typical regimen lasting 7 to 14 days, depending on the desired whitening outcome.

During the application period, the strip remains in place through passive adhesion, gradually releasing the enzymatic and botanical active agents onto the tooth surface. Salivary hydration facilitates diffusion of the actives into interproximal and occlusal crevices, enhancing coverage. The release kinetics of the active compounds are governed by the matrix composition of the substrate, which is formulated to permit sustained, controlled delivery without burst release or premature depletion. Upon completion of the treatment interval, the strip may be removed manually and discarded, with no need for rinsing or brushing unless desired for comfort.

The whitening process is cumulative, with incremental improvements in shade perceptibility occurring over multiple applications. Clinical and consumer-facing studies utilizing standardized tooth shade guides (e.g., VITA Classical or VITA 3D-Master) have demonstrated that the invention may achieve whitening improvements of up to five shades over the course of a standard 14-day regimen, without reported sensitivity or adverse oral tissue reactions. Notably, the enzymatic whitening approach provides more gradual, uniform shade changes compared to peroxide-based bleaching, with reduced incidence of mottling or uneven coloration.

From a manufacturing standpoint, the strips may be fabricated using a continuous roll-to-roll process incorporating casting, drying, cutting, and packaging steps in an aseptic environment. The formulation is applied via a precision coating system—such as slot-die, doctor blade, or curtain coating—onto the substrate film, followed by controlled drying at low temperatures to preserve enzyme activity. Drying temperatures are maintained between 25 °C and 40 °C, depending on the thermal stability of the active ingredients. Once dried, the strips are cut to specification using rotary or flat-bed die-cutting systems and are then sealed into individual foil pouches using inert gas flushing or desiccant chambers to minimize moisture ingress.

The entire production process adheres to cGMP (current Good Manufacturing Practices) guidelines and may be implemented under ISO 22716 standards for cosmetic products or ISO 13485 standards for medical devices, depending on the jurisdiction and regulatory classification. Enzymatic raw materials are sourced in compliance with food-or pharmaceutical-grade purity specifications and are validated for microbial safety, stability, and bioactivity prior to formulation. Quality assurance protocols include batch-level enzymatic activity assays, microbial testing, pH verification, and visual inspection of strip uniformity.

Shelf-life stability testing, performed under accelerated and real-time storage conditions, has demonstrated that the product retains over 90% of its enzymatic activity after 18 months when stored in ambient conditions (25 °C, 60% RH) in its original packaging. Optional enhancements, such as UV-blocking foil laminates or oxygen scavengers, may be included in the primary packaging system to further extend product longevity and efficacy.

Accordingly, the method of use and manufacturing practices associated with the present invention support a commercially scalable, consumer-friendly whitening system that differentiates itself from peroxide-based competitors through its ease of use, natural formulation, and enhanced safety profile. These factors collectively reinforce the invention's commercial viability and patentability as a novel advancement in the field of dental aesthetics and oral care delivery systems.

### DETAILED DESCRIPTION OF FIGURES

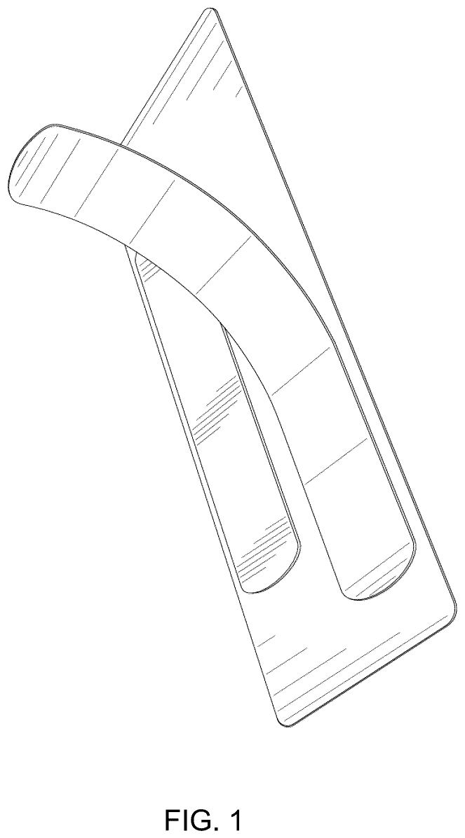

FIG. 1 depicts an overhead (planar) view of the whitening strip assembly, comprising an upper strip (101) and a lower strip (103), each configured to conform anatomically to the maxillary and mandibular dental arches, respectively. The strips are shown in their pre-application form, prior to contact with saliva or oral surfaces. Each strip exhibits a contoured geometry, optionally including flared lateral ends (105) for coverage of posterior teeth and a central narrowing (107) to accommodate labial curvature. The active whitening composition (109) is uniformly or selectively distributed across the interior-facing surface of each strip and may be visually distinguishable from the substrate material by color, texture, or translucency. The strips are sized and shaped to ensure ergonomic placement and full coverage of visible dentition during use.

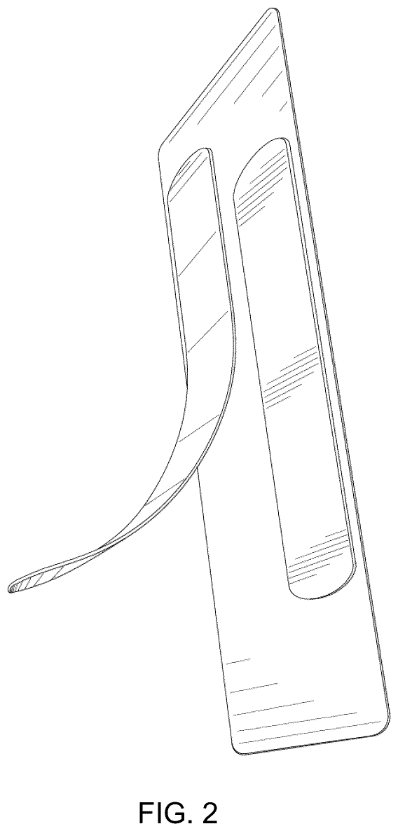

FIG. 2 presents a side perspective view illustrating the removal of the whitening strip (201) from a storage liner or protective backing layer (203). The strip includes a flexible substrate (205) having an interior surface on which the whitening composition (207) is disposed. During storage, the formulation-carrying surface faces inward against the liner to prevent premature exposure to air or moisture. As illustrated, a user or automated device may lift one end of the strip (209) to initiate removal, resulting in a partial separation angle (211) between the strip and the liner. The backing layer is typically composed of a non-stick material such as polyethylene-coated paper or silicone-treated film that permits easy separation without disturbing the integrity of the applied composition. This configuration ensures that the active agents remain protected and sterile until immediately prior to intraoral application.

What is claimed is:

1. A tooth whitening strip comprising:
a. a flexible, biocompatible substrate; and
a whitening composition disposed on or within the substrate, the whitening composition comprising:
(i) papain;
(ii) bromelain;
(iii) aloe vera extract;
(iv) coconut oil; and
(v) green tea extract;
wherein the composition is substantially free of peroxide-based whitening agents, and wherein the strip is configured to adhere to dental surfaces through saliva-activated hydration of the substrate, without the use of synthetic adhesives.

2. The tooth whitening strip of claim 1, wherein the papain and bromelain are present in a combined concentration ranging from approximately 0.05% to 2.0% by weight of the total composition.

3. The tooth whitening strip of claim 1, wherein the green tea extract comprises epigallocatechin gallate (EGCG) in a concentration ranging from approximately 0.1% to 3.0% by weight.

4. The tooth whitening strip of claim 1, wherein the substrate comprises hydroxypropyl methylcellulose (HPMC), carboxymethylcellulose (CMC), polyvinyl alcohol (PVA), or a combination thereof.

5. The tooth whitening strip of claim 1, wherein the substrate is microporous and configured to allow penetration of saliva into the formulation layer.

6. The tooth whitening strip of claim 1, wherein the aloe vera extract and coconut oil are present as emollients and humectants in a total concentration of approximately 1.0% to 10.0% by weight.

7. The tooth whitening strip of claim 1, further comprising a pH stabilizer selected from the group consisting of sodium citrate and potassium bicarbonate, wherein the formulation maintains a pH between 6.2 and 7.4.

8. The tooth whitening strip of claim 1, wherein the formulation is applied via solvent casting or lamination, and is dried under controlled temperature conditions to preserve enzymatic activity.

9. A method of whitening teeth, comprising:
(a) providing the tooth whitening strip of claim 1;
(b) applying the strip to a user's upper or lower dental arch;
(c) allowing the strip to remain in place for a period of 15 to 30 minutes; and
(d) removing the strip without the need for rinsing or brushing,
wherein the enzymatic whitening composition removes extrinsic stains from the teeth without the use of peroxide and without causing tooth sensitivity.

10. The method of claim 9, wherein step (c) includes exposure of the strip to saliva, thereby activating adhesion of the strip to the teeth through hydration of the substrate.
